(19) United States
(12) Patent Application Publication
(10) Pub. No.: US 2026/0272509 A1
(43) Pub. Date: Sep. 17, 2026

(54) UTERINE HEMORRHAGE CONTROLLING DEVICE

(71) Applicant: Alydia Health, Inc., Menlo Park, CA (US)

(72) Inventors: Peter Calvin Costello, Providence, RI (US); Melissa Christine Dols, Jersey City, NJ (US); Dustin Gaidos, Providence, RI (US); James David Lorek, Plymouth Meeting, PA (US); Daniel P. Smith, Providence, RI (US)

(21) Appl. No.: 19/563,581

(22) Filed: Mar. 11, 2026

Related U.S. Application Data
(60) Provisional application No. 63/770,108, filed on Mar. 11, 2025.
(60) Provisional application No. 63/877,441, filed on Sep. 8, 2025.

(51) Int. Cl. A61B 17/42

(52) U.S. Cl. CPC A61B 17/42; A61B 2017/4216; A61B 2217/005

(57) ABSTRACT

A uterine hemorrhage controlling device includes a suction tip having a plurality of tip openings connected to a distal end of an elongated shaft. A seating element is connected to the shaft at a location proximal to the suction tip. The seating element extends radially from the elongated shaft and comprises a flexible or inflatable portion configured to seat the device within the vagina to facilitate the creation of a negative pressure within the uterine cavity. The suction tip is positioned inside the uterine cavity and the proximal end of the elongated shaft is connected to a source of negative pressure. The negative pressure is delivered from outside the vagina through a lumen in the shaft to a lumen in the suction tip and then through the tip openings to apply a negative pressure to the interior of the uterine cavity to reduce uterine hemorrhaging.

### CROSS-REFERENCE TO RELATED APPLICATIONS

This application claims the benefit under 35 U.S.C. § 119(e) of U.S. Provisional Patent Application Serial Nos. 63/770,108 and 63/877,441, filed on Mar. 11, 2025 and Sep. 8, 2025, respectively, the disclosure of each of which is hereby incorporated herein in its entirety by this reference.

### TECHNICAL FIELD

This application generally relates to medical devices and, in particular, to medical systems, devices, and methods for treating postpartum hemorrhage and excessive uterine bleeding.

### BACKGROUND

Postpartum hemorrhage, defined as excessive blood loss after birth, is the leading cause of maternal death in the world, claiming the lives of over 125,000 mothers every year. Inability to control postpartum bleeding can require a woman to receive multiple blood transfusions, and in severe cases, a full hysterectomy or death. Therefore, it is desirable to control such postpartum bleeding, if possible, at its onset. The cause of postpartum hemorrhage, in approximately 80% of cases, is uterine atony, which is the inability of the woman's uterus to contract after delivering the child. Risk factors for uterine atony include prolonged stage of labor, preeclampsia, and multiparity. Accordingly, a system that is able to rapidly induce uterine contraction, which may reduce or entirely stop uterine hemorrhaging, is needed.

### BRIEF DESCRIPTION OF THE SEVERAL VIEWS OF THE DRAWINGS

The various advantages of the embodiments of the present disclosure will become apparent to one skilled in the art by reading the following specification and appended claims, and by referencing the following drawings in which:

FIG. 1 is a top perspective view of an anti-hemorrhage device, according to one or more embodiments;

FIG. 2 is a side elevational view of the device of FIG. 1, according to one or more embodiments;

FIG. 3 is a top view of the device of FIG. 1, according to one or more embodiments;

FIG. 4 is a cross-sectional view of the device taken along line 4-4 of FIG. 2, according to one or more embodiments;

FIG. 5 is a cross-sectional view of the device taken along line 5-5 of FIG. 3, according to one or more embodiments;

FIG. 6 is a top perspective view of a seating element of the device of FIG. 1, according to one or more embodiments;

FIG. 7 is a proximal end view of the seating element of FIG. 6, according to one or more embodiments;

FIG. 8 is a cross-sectional view of the seating element taken along line 8-8 of FIG. 7, according to one or more embodiments;

FIG. 9 is a cross-sectional view of the seating element taken along line 9-9 of FIG. 7, according to one or more embodiments;

FIG. 10 is a top perspective view of an anti-hemorrhage device, according to one or more embodiments;

FIG. 11 is a side elevational view of the device of FIG. 10, according to one or more embodiments;

FIG. 12 is a top view of the device of FIG. 10, according to one or more embodiments;

FIG. 13A is a side elevational view of a hub of the device of FIG. 10, according to one or more embodiments;

FIG. 13B is a top view of a hub of FIG. 13A, according to one or more embodiments;

FIG. 13C is cross-sectional view taken along line 13C-13C of the hub of FIG. 13B, according to one or more embodiments;

FIG. 14A is a top view of an inflatable seating element of the device of FIG. 10, according to one or more embodiments;

FIG. 14B is a cross-sectional view taken along line 14B-14B of the inflatable seating element of FIG. 14A, according to one or more embodiments;

FIG. 15 is a cross-sectional view taken along line 15-15 of the device of FIG. 12, according to one or more embodiments;

FIG. 16 is a detailed view of section 16 of the device of FIG. 15, according to one or more embodiments;

FIG. 17 is a top perspective view of an inflatable seating element, according to one or more embodiments;

FIG. 18 is an elevational end view of the inflatable seating element of FIG. 18, according to one or more embodiments;

FIG. 19 is a cross-sectional view taken along line 19-19 of the inflatable seating element of FIG. 18, according to one or more embodiments;

FIG. 20 is a top perspective view of a hub, according to one or more embodiments;

FIG. 21 is a top perspective view of a clip, according to one or more embodiments;

FIG. 22 is an exploded view of an insertable device, according to one or more embodiments;

FIG. 23 is a top perspective view of a tip of an insertable device, according to one or more embodiments;

FIG. 24 is a side elevational view of the tip of FIG. 23, according to one or more embodiments;

FIG. 25 is a top view of the tip of FIG. 23, according to one or more embodiments;

FIG. 26 is an end view of the tip of FIG. 23, according to one or more embodiments;

FIG. 27 is a cross-sectional view taken along line 27-27 of FIG. 24, according to one or more embodiments;

FIG. 28 is a cross-sectional view taken along line 28-28 of FIG. 25, according to one or more embodiments;

FIG. 29 is a cross-sectional view taken along line 29-29 of the tip of FIG. 25, according to one or more embodiments;

FIG. 30 is a cross-sectional view taken along line 30-30 of the tip of FIG. 25, according to one or more embodiments;

FIG. 31 is a top perspective view of a tip of an insertable device, according to one or more embodiments;

FIG. 32 is a side elevational view of the tip of FIG. 25, according to one or more embodiments;

FIG. 33 is a top view of the tip of FIG. 25, according to one or more embodiments;

FIG. 34 is an end view of the tip of FIG. 25, according to one or more embodiments;

FIG. 35 is a cross-sectional view taken along line 35-35 of the tip of FIG. 32, according to one or more embodiments;

FIG. 36 is a cross-sectional view taken along line 36-36 of the tip of FIG. 33, according to one or more embodiments; and

FIG. 37 is a cross-sectional view taken along line 37-37 of the tip of FIG. 33, according to one or more embodiments.

### DETAILED DESCRIPTION

Described herein are uterine hemorrhage controlling devices, systems, and methods that create a vacuum within the uterine space while evacuating blood and/or clots therefrom. The vacuum can advantageously encourage physical collapse of the uterine space, leading to inducement of tone in the uterus to affect control of the hemorrhage.

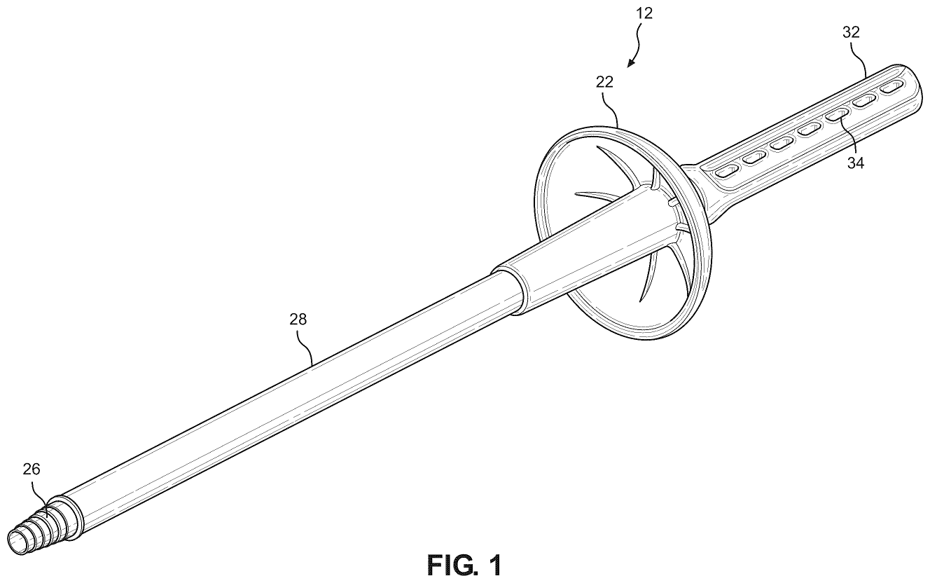

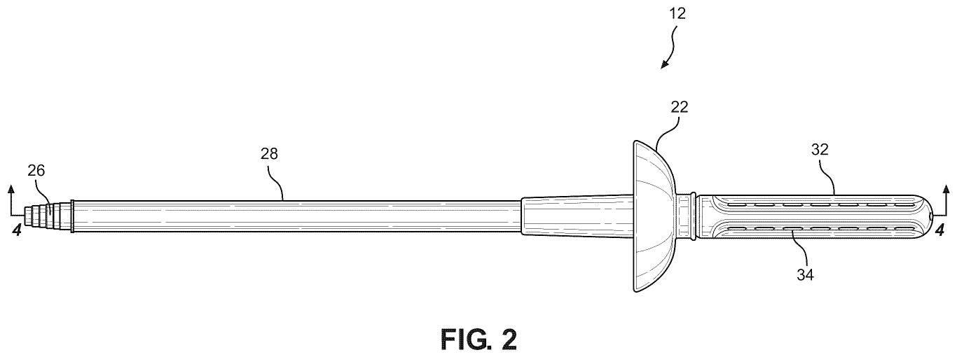

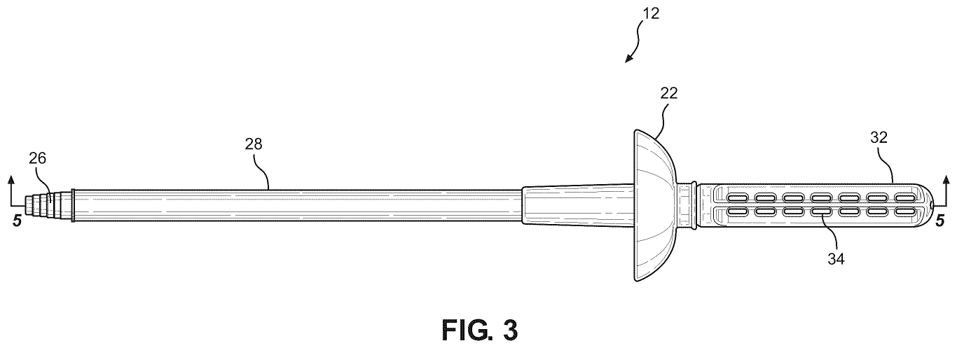

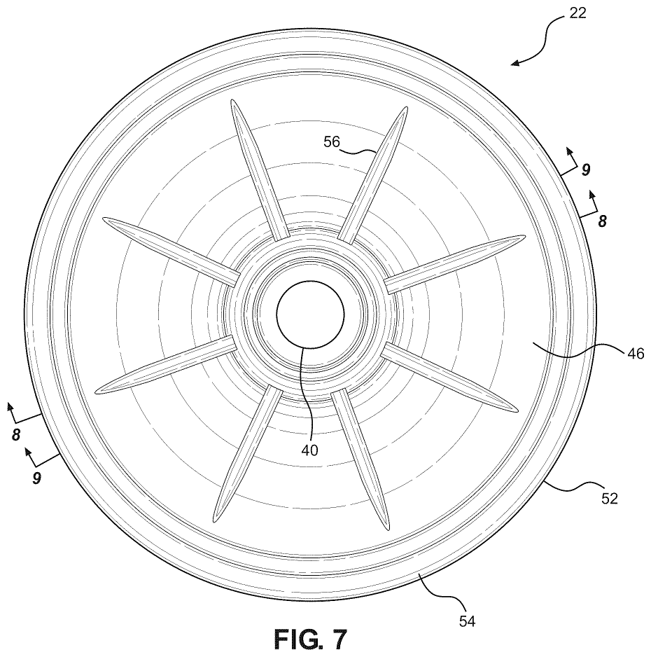

FIGS. 1-9 illustrate an exemplary insertable device 12 that is configured to be inserted into a uterus for controlling uterine hemorrhaging. The insertable device 12 includes a tip 32 connected to and extending distally from a seating element 22 that is connected to a shaft 28 having a connector 26 at a proximal end. The connector 26 at the proximal end is connectable to a source of negative pressure such as a pump via connection tubing. The tip 32 of the insertable device 12 is delivered transvaginally (through the vagina) such that it is positioned within the uterus while a proximal portion of the insertable device 12, including a portion of the shaft 28, remains external to the uterus. When the source of negative pressure is activated, vacuum is delivered via the attached connection tubing to the shaft 28, to a plurality of openings 34 in the tip 32, and to the uterine cavity.

The seating element 22 extends radially outwardly from the shaft 28 and contacts the surrounding anatomy, such as the vaginal canal. The seating element 22 may be positioned anywhere along the vagina including but not limited to between the vaginal and cervical openings. The seating element 22 reduces or completely blocks the area through which air/fluid may flow into or out of the uterus serving as an abutment or bulkhead that facilitates a pressure change within the uterus. A negative pressure or vacuum is achieved within the uterus as air/fluid is pulled out of the uterine cavity through the tip 32 at a rate faster than air entering the uterine cavity. The seating element 22 enhances the ability in establishing a low pressure within the uterus. The degree to which low pressure is established and/or the rate by which low pressure is established by the seating element 22 contacting surrounding tissue and/or at least partially sealing against the surrounding anatomy, leading to evacuation of air and/or blood from the uterus through the tip 32 and shaft 28.

A collection container may be attached to the connection tubing to collect blood and debris extracted by the suction. With the application of negative pressure for a period of time, the pressure inside the uterus drops to lower than atmospheric pressure outside of the uterus. The user can monitor and regulate the source of negative pressure to provide and maintain the desired amount of vacuum pressure for desired performance such as between approximately 60-150 mmHg. The reduced pressure inside the uterus facilitates tamponade, arterial vessel constriction, and contractile movement of the uterine wall by providing a uniform mechanical stimulus. In addition, a user may monitor and measure the amount of blood loss during the procedure and determine whether, when and/or to what extent uterine contraction has occurred.

The system advantageously functions to significantly reduce or entirely stop uterine hemorrhaging. Controlling uterine hemorrhaging substantially reduces the total blood lost from the uterus and may reduce a woman's need for a blood transfusion or a hysterectomy. This is particularly important after childbirth when a woman may experience uterine atony wherein the uterus fails to contract. The system facilitates contraction of the uterus by creating a pressure change within the uterus. Changing the pressure to generate a negative pressure within the uterus results in a uniform mechanical stimulus to the uterine wall in order to facilitate tamponade and contractile movement of the tissue.

The insertable device 12 includes a shaft 28 defining a shaft lumen 30. The shaft lumen 30 extends along the length of the shaft 28 from a proximal end to a distal end. The tip 32 includes a plurality of tip openings 34. The tip openings 34 are in fluidic communication with the tip lumen 33 which is in fluidic communication with the shaft lumen 30. In one variation, the shaft lumen 30 may be divided such as with a septum forming one or more channels extending along the length of shaft lumen 30. A divided shaft lumen 30 can provide a redundancy and continued functionality of negative pressure generation in the event one channel is clogged with debris. The shaft 28 is flexible and relatively more rigid than the connection tubing to facilitate handling of the device 12. The distal end of the shaft 28 is connected to a proximal end of the seating element 22.

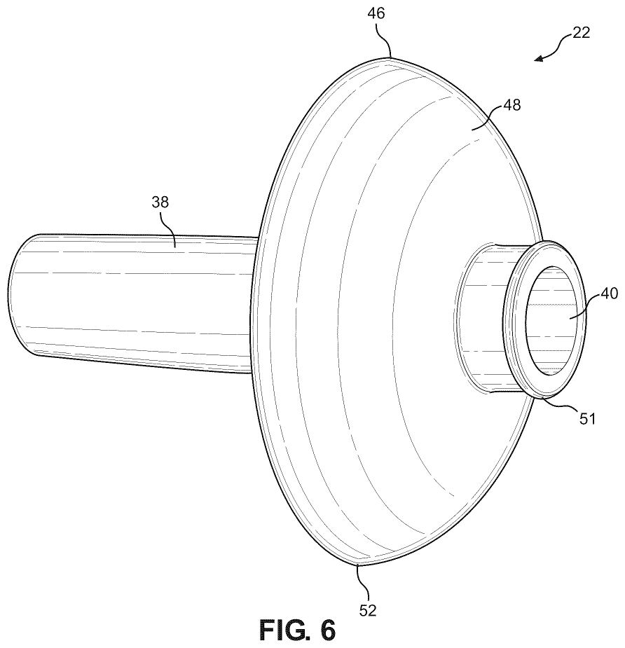

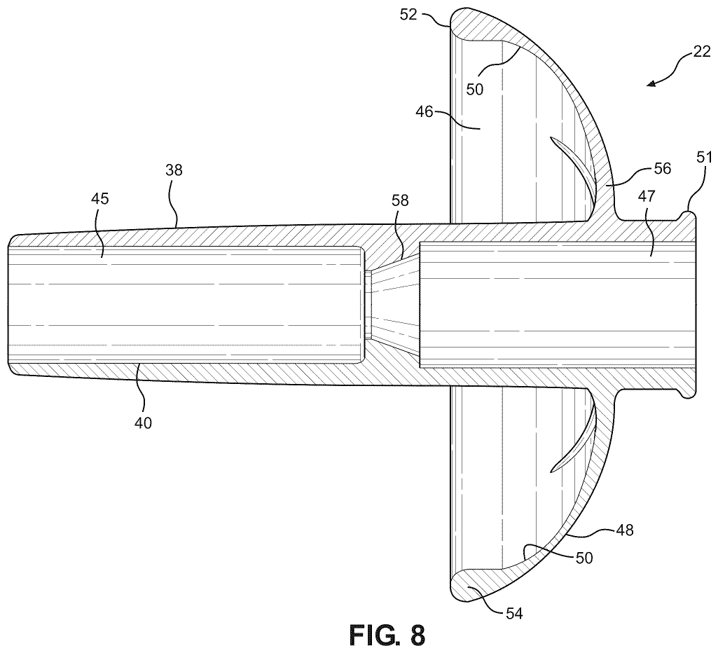

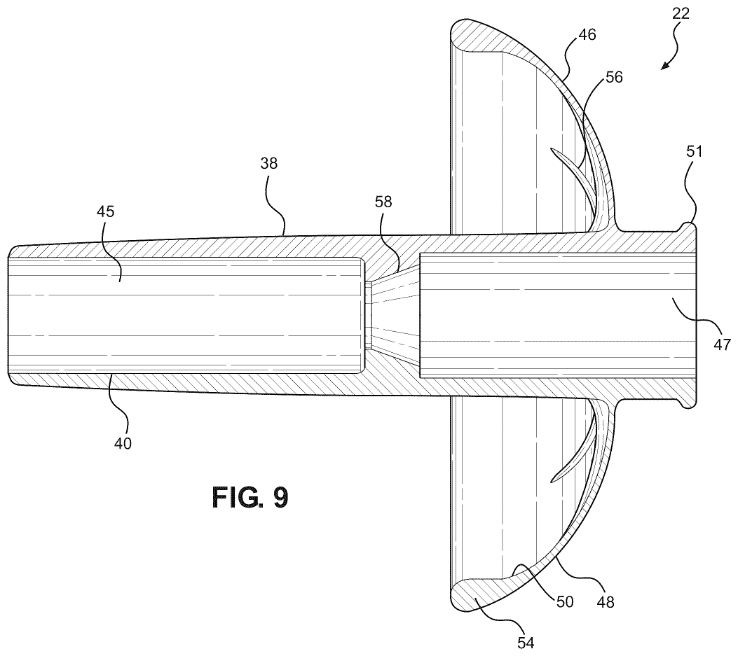

With particular reference to FIGS. 6-9, the seating element 22 includes a stem 38 defining a stem lumen 40. The stem lumen 40 extends coaxially along a longitudinal axis between an opening at a proximal end and an opening at a distal end. The proximal end of the stem lumen 40 is sized and configured to receive a distal end of the shaft 28. The seating element 22 includes a flared portion 46 that extends radially outwardly generally perpendicular to the longitudinal axis. The seating element 22 has an outer diameter that is larger than the outer diameter of the shaft 28. In one variation, the outer diameter of the seating element 22 is approximately 7 cm. The flared portion 46 has a smooth, convex distal surface 48 interconnected to a proximal surface 50 at a circumferential edge/perimeter 52. The convex distal surface 48 is opposite to the concave proximal surface 50 when in a normal, unbiased configuration. The edge/perimeter 52 forms an atraumatic circular shape and defines a circumferential bead 54 having an increased material thickness. The flared portion 46 forms a funnel-like, cup-like, or umbrella-like shape and includes several radially extending ribs 56 on the proximal surface 50. The ribs 56 are formed symmetrically around the stem 38 in a star-like pattern. Each rib 56 is formed on the concave proximal surface 50 and has an increased material thickness relative to surrounding adjacent areas of the flared portion 46 without ribs 56. Each rib 56 has smooth atraumatic rounded shape that advantageously prevents tissue trauma when the device 12 is removed. The ribs 56 advantageously provide rigidity and structural shape integrity to the flared portion 46. The thickness of each rib 56 is greatest near the stem lumen 40 and decreases with radial distance away from the longitudinal axis. As can be seen in a cross-section of the seating element 22 taken through the ribs 56 in FIG. 8, the cross-sectional thickness of the flared portion 46 decreases with radial distance from the longitudinal axis and then increases at the edge 52 to form the bead 54. As can be seen in a cross-section of the seating element 22 taken through areas of the flared portion 46 without ribs 56 in FIG. 9, the thickness of the flared portion 46 is relatively thin and constant and then increases at the bead 54. This ribbed geometry of the flared portion 46 advantageously provides spring back of the flared portion 46 when it is deflected/collapsed to facilitate ease of insertion and removal of the device 12 and also, advantageously permits increased conformance of the flared portion 46 against the anatomy when pressure is applied, thereby, improving sealing and maintenance of a negative pressure. The seating element 22 is made of a flexible, resilient and soft elastomeric material having a durometer in the range of 40 A to 60 A such as silicone rubber, thermoplastic elastomer, or thermoplastic urethane. The soft material prevents tissue trauma while providing sufficient flexibility and stiffness for usability. The material and rib geometry that includes multiple concavely-curved ribs arranged around a longitudinal axis impart the seating element 22 with a shape memory biased to its original shape.

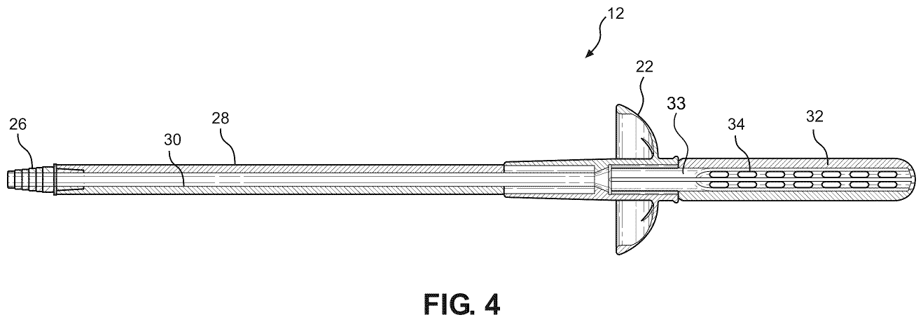

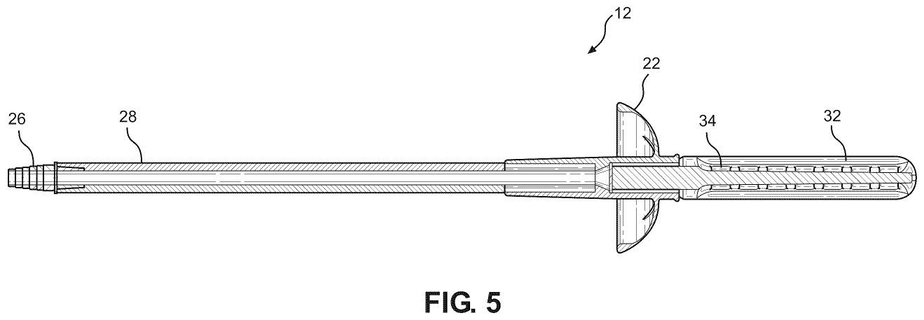

With particular reference to FIGS. 8 and 9, the stem lumen 40 includes a proximal portion 45 sized and configured to receive the distal end of the shaft 28 and a distal portion 47 sized and configured to receive a proximal end of the tip 32 interconnected by a frustoconical interconnecting portion 58. In one variation, the interconnecting portion 58 has a smaller inner diameter at the proximal end which increases to a larger diameter at the distal end of the interconnecting portion 58. As can be seen in FIGS. 4 and 5, when the distal end of the shaft 28 is inserted into the proximal portion 45, the inner diameter of the shaft 28 is approximately equal to the proximal inner diameter of the frustoconical interconnecting portion 58 and when the proximal end of the tip 32 is inserted into the distal portion 47, the distal inner diameter of the frustoconical interconnecting portion 58 is approximately equal to the inner diameter of the proximal end of the tip 32 to advantageously create a smooth flow path transition from the tip 32 to the shaft 28 and advantageously prevent clogging or backing up of debris during suction.

The stem 38 is tapered such that the outer diameter of the stem 38 decreases with distance from the flared portion 46 to the proximal end. This tapered proximal end of the stem 38 forms a smooth transition to the shaft 28 to prevent snag points, tissue trauma and facilitate smooth insertion and removal of the device 12. The stem 38 extends distally from the flared portion 46 to a distal end that includes a circumferential ridge 51. The ridge 51 has an outer diameter slightly larger than the outer of the stem 38 such that the ridge 51 protrudes radially above the outer surface of the stem 38 and an outer surface of the tip 32. When the tip 32 is connected to the seating element 22, the ridge 51 advantageously defines a tactile intersection between the seating element 22 and the tip 32 that helps the user to locate and guide the tip 32 into position.

The device 12 is inserted into the vaginal canal with the tip 32 leading such that the convex distal surface 48 faces the uterus. During insertion, at least part of the flared portion 46 may become deflected. Since the flared portion 46 is biased towards an undeflected configuration, the seating element 22 will spring back towards its full original shape or may remain partially deflected against patient anatomy contacting the vaginal canal or cervical canal in a circumferential manner to facilitate formation of a reduced pressure zone within the uterus. The seating element 22 engages vaginal tissue to limit the loss of vacuum. Any part of the flared portion 46, such as the bead 54, may press against or make continuous radial contact with tissue of the vaginal canal or cervical wall to maintain a negative pressure inside uterus. The shape memory and stiffness of the flared portion 46, due, in part, to the rib geometry, allows the flared portion 46 to spring back and maintain its shape and position perpendicular to the longitudinal axis. Also, the relatively thicker bead 54 around outer rim helps to maintain the hoop shape and structure for improved performance. When the device 12 is inserted or removed, the rib geometry of the flared portion 46 may flex and readily invert forward and backward as the device 12 is moved along the longitudinal axis. The material flexibility, smooth convex distal surface 48, rounded bead 54 and ribs 56 advantageously reduce the applied force on surrounding tissues to prevent tissue trauma.

The user may intentionally invert the flared portion 46 prior to insertion. The flared portion 46 may be converted between the inverted and normal configurations by pulling or pushing the device 12 along a longitudinal direction to flip the flared portion 46. The ability of the seating element 22 to invert into an inverted configuration provides an additional ability for the seating element 22 to contact tissue for facilitating maintaining a vacuum in the uterus. When in a partially inverted configuration, the flared portion 46 may adopt a larger outer diameter and the proximal surface 50 may advantageously also serve to make contact greater contact with tissue for more complete sealing of the uterus. A complete seal of the uterine cavity may be achieved but is not necessary to facilitate a creation of at least a partial vacuum and achieve a therapeutic benefit. The flexibility of the flared portion 46 to take an inverted configuration advantageously provides mechanical compliance of the flared portion 46 against anatomical structures thereby promoting the creation of a vacuum within the uterus when negative pressure is applied to the distal end of the device 12. Also, inversion of the flared portion 46 advantageously prevents injury to tissue when the device 12 is being inserted and removed. Furthermore, the bead 54 provides a smooth atraumatic surface for the prevention of injury. Also, the increased thickness of the bead 54 advantageously provides more surface area contact for improved sealing.

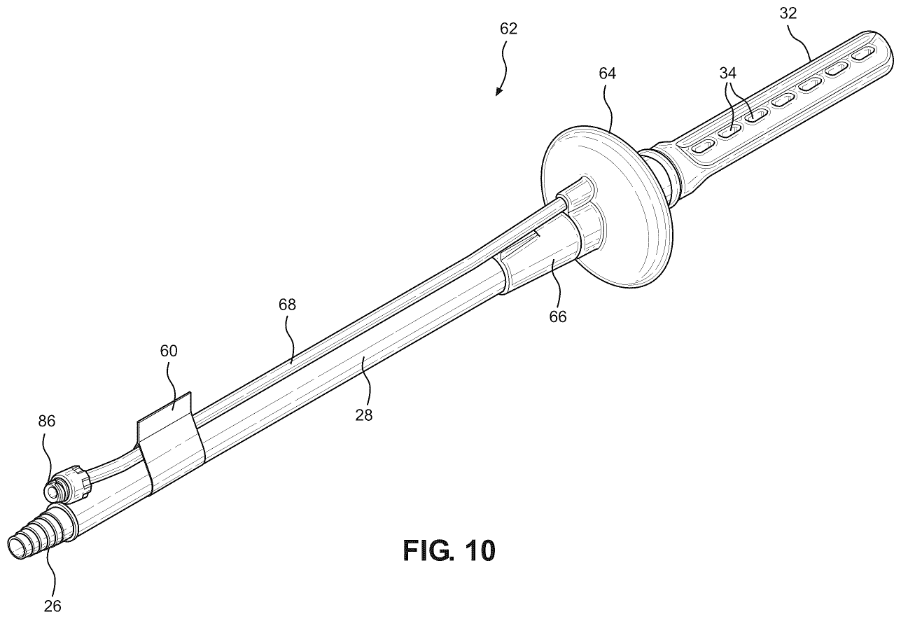

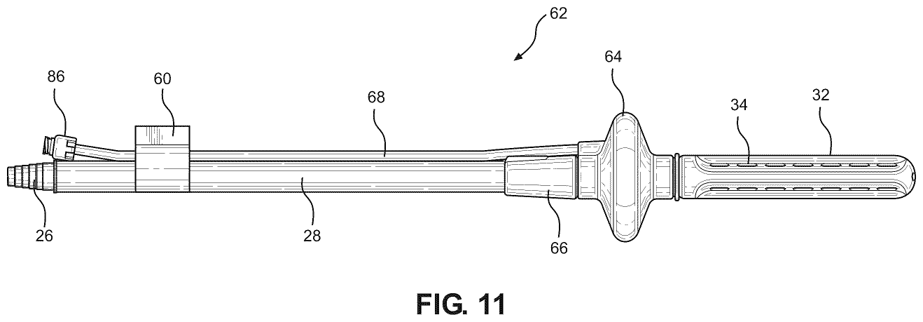

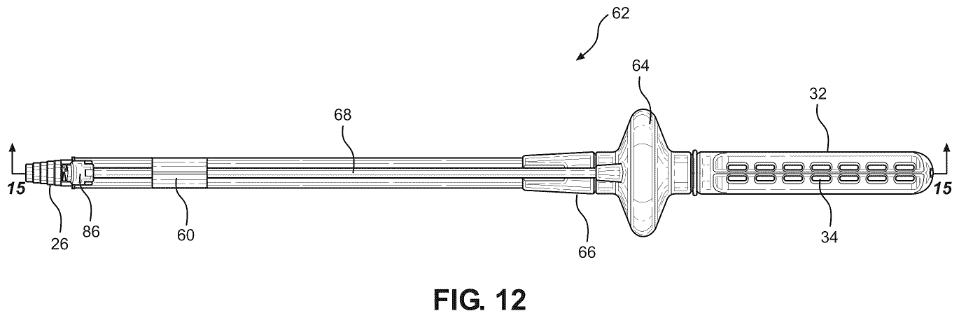

FIGS. 10-16 illustrate another embodiment of an insertable device 62 for controlling uterine hemorrhaging wherein like reference numbers are used to describe like parts. The insertable device 62 is similar to the device of FIGS. 1-9 being configured to be inserted into the uterus for controlling uterine hemorrhaging. The insertable device 62 includes a shaft 28. A connector 26 is connected to the proximal end of the shaft 28. The distal end of the shaft 28 is connected to the proximal end of a hub 66. The distal end of the hub 66 is connected to a tip 32 having a plurality of openings 34. An inflatable seating element 64 is mounted and connected to the hub 66. An inflation tube 68 for inflating the inflatable seating element 64 is connected to the inflatable seating element 64. The proximal end of the inflation tube 68 includes a connector 86. A fastener 60 holds the proximal end of the inflation tube 68 connected to shaft 28. The connector 26 at the proximal end of the shaft 28 is connected to a source of negative pressure such as a pump via connection tubing. The insertable device 62 is inserted transvaginally such that the tip 32 is positioned within the uterus, inflation fluid is delivered into the inflatable seating element 64 and expanded to contact vaginal anatomy and vacuum is delivered from the source of negative pressure through the connection tubing, shaft 28, hub 66, tip 32 and tip openings 34 to arrest uterine hemorrhaging.

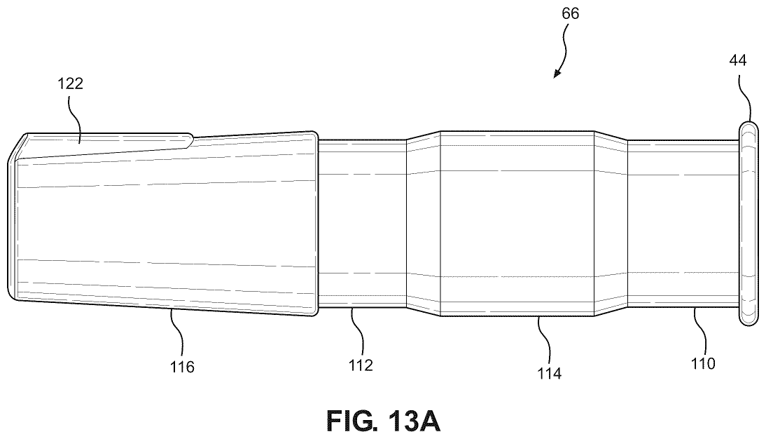

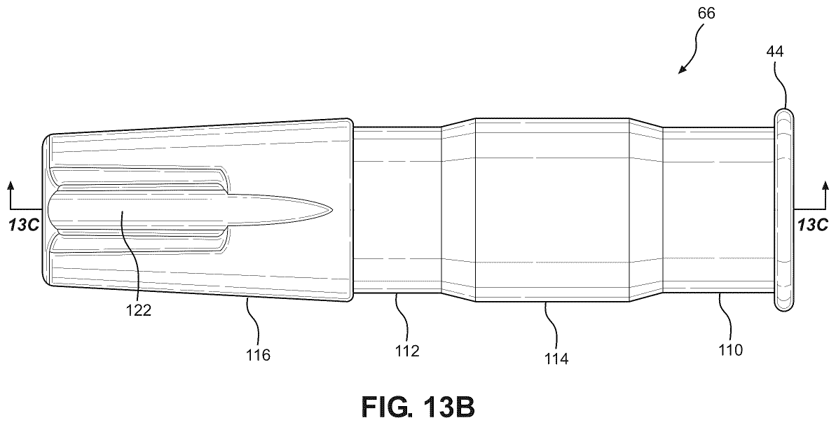

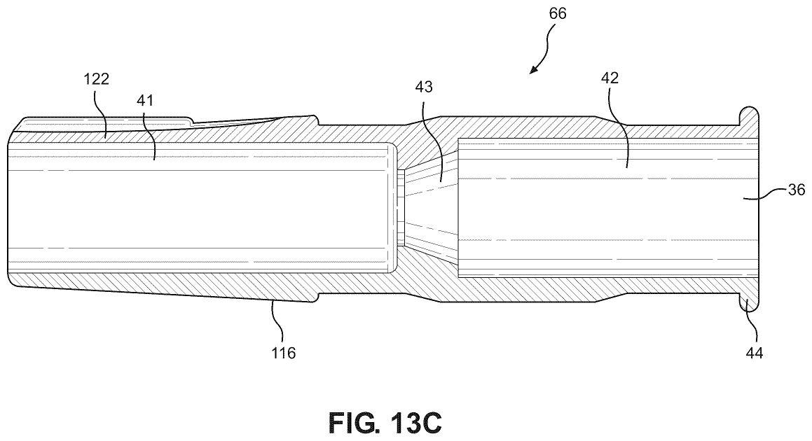

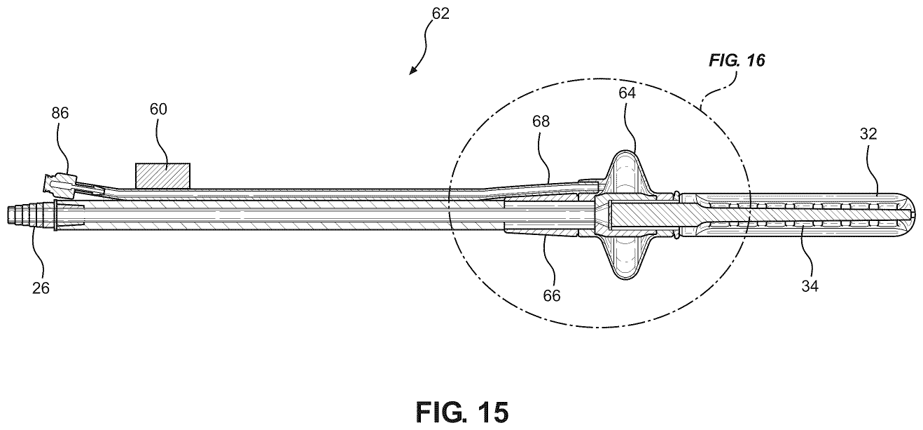

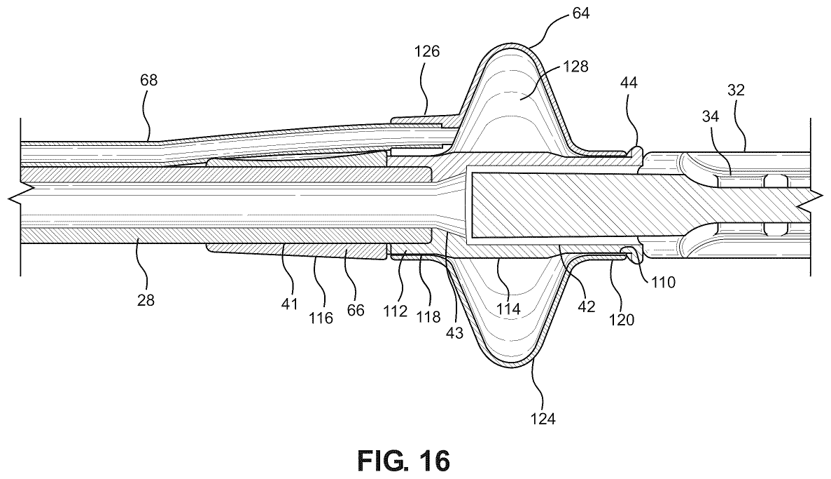

With particular reference to FIGS. 13A-13C, the hub 66 includes a hub lumen 36 extending between an open proximal end and an open distal end. The hub lumen 36 includes a proximal portion 41 sized and configured to receive the distal end of the shaft 28 and a distal portion 42 sized and configured to receive a proximal end of the tip 32. The proximal portion 41 and the distal portion 42 are interconnected by a hub transition portion 43. The transition portion 43 of the hub lumen 36 has a frustoconical shape having a smaller inner diameter at the proximal end that increases to a larger diameter at the distal end of the transition portion 43. As can be seen in FIGS. 15 and 16, when the distal end of the shaft 28 is inserted into the proximal portion 41 of the hub 66, the inner diameter of the shaft 28 is approximately equal to the proximal inner diameter of the frustoconical transition portion 43 and when the proximal end of the tip 32 is inserted into the distal portion 42, the distal inner diameter of the frustoconical interconnecting portion 58 is approximately equal to the inner diameter of the proximal end of the tip 32 to advantageously create a smooth flow path transition from the tip 32 to the shaft 28 and advantageously prevent clogging or backing up of flow and debris during suction.

Furthermore, the hub 66 is tapered such that the outer diameter of the hub 66 at a location proximal to the inflatable seating element 64 decreases in a proximal direction along a longitudinal axis defining a tapered proximal portion 116 of the hub 66. The outer diameter of the hub 66 directly proximally adjacent to the inflatable seating element 64 has approximately the same outer diameter as the proximal end of inflatable seating element 64 to form a smooth outer transition between the inflatable seating element 64 and the hub 66. The tapered proximal portion 116 of the hub 66 further forms a smooth transition to the shaft 28 to prevent snag points, tissue trauma and facilitate smooth insertion and removal of the device 62. The distal end of the hub 66 includes a circumferential ridge 44. The ridge 44 has an outer diameter that is slightly larger than the outer diameter of the distal portion of the hub 66 such that the ridge 44 protrudes radially above the outer surface of the distal end of inflatable seating element 64 and above an outer surface of the proximal end of the tip 32. When the proximal end of the tip 32 is inserted into the distal portion 42 of the hub lumen 36 of the hub 66, the ridge 44 advantageously defines a tactile intersection between the distal end of the inflatable seating element 64 and the tip 32 that assists the user in locating and guiding the tip 32 into position.

The outer surface of the hub 66 defines a seating location for seating the inflatable seating element 64. The seating location includes a distal seating section 110 and proximal seating section 112 separated by a central seating section 114. The distal seating section 110 is a cylindrical portion of the hub 66 that has an outer diameter that is smaller than the outer diameter of the tapered proximal portion 116 and the central seating section 114 at interfaces therewith. Similarly, the proximal seating section 110 is a cylindrical portion of the hub that has an outer diameter that is smaller than the outer diameter of the central seating section 114 and the ridge 44 at interfaces therewith. The distal seating section 110 and proximal seating section 112 serve as attachment locations for a distal cylindrical portion 120 and proximal cylindrical portion 118 of the inflatable seating element 64, respectively. As can be seen in FIG. 16, when the inflatable seating element 64 is attached to the hub 66, the outer surface of the proximal cylindrical portion 118 of the inflatable seating element 64 is substantially flush with the tapered proximal portion 116 of the hub 66 to form a smooth transition. When the distal cylindrical portion 120 of the inflatable seating element 64 is attached to the distal seating section 110, the outer surface of the distal cylindrical portion 120 of the hub 66 will have an outer diameter smaller than the ridge 44 such that the ridge 44 can provide tactile feedback to the user.

The tapered proximal portion 116 includes a channel 122 for receiving the inflation tube 68. The channel 122 is a concave indentation extending parallel to the longitudinal axis of the proximal tapered portion and is sized and configured to seat the inflation tube 68. The channel 122 allows for placement of the inflation tube 68 closer to the shaft 28 to provide less interference and facilitate deployment of the device 62. The hub 66 is made from any suitable material such a soft elastomer, silicone rubber, thermoplastic elastomer, or thermoplastic urethane. A soft elastomer having a durometer in the range of approximately 40-60 A provides sufficient flexibility and stiffness and prevents tissue trauma.

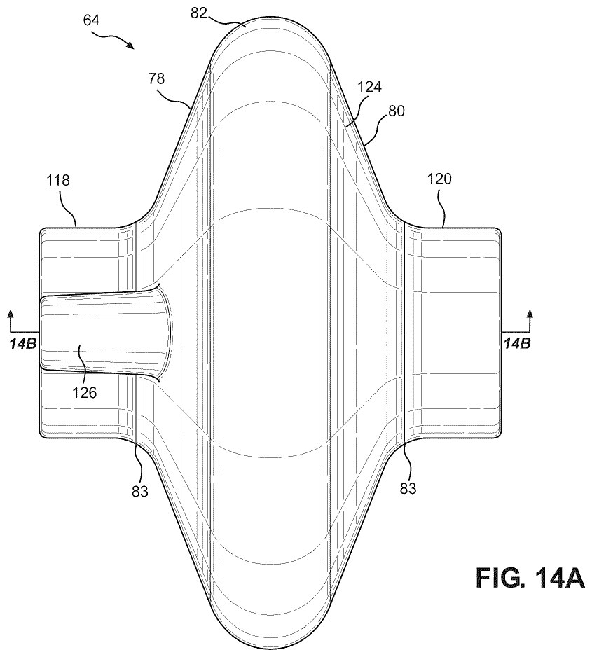

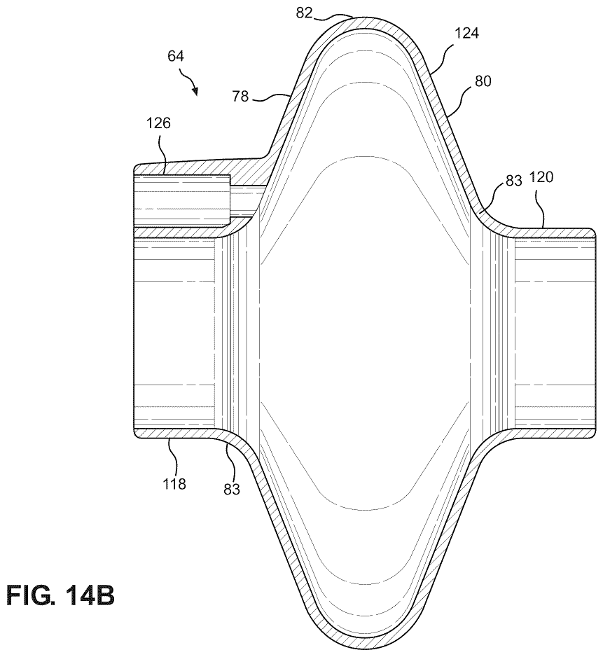

FIGS. 14A and 14B illustrate the inflatable seating element 64. The inflatable seating element 64 includes a proximal cylindrical portion 118 interconnected to a distal cylindrical portion 120 by a central inflatable portion 124. An inflation port 126 is integrally formed on the proximal side of the inflatable portion 124. The inflation port 126 is sized and configured to receive the inflation tube 68 and place it in fluidic communication with the interior of the inflatable portion 124 for inflation and deflation of the inflatable seating element 64. The distal cylindrical portion 120 defines a distal opening that is sized and configured for receiving and encompassing the distal seating section 110 of the hub 66 and being secured thereto. The proximal cylindrical portion 118 defines a proximal opening that is sized and configured for receiving and encompassing the proximal seating section 112 of the hub 66 and being secured thereto. When attached to the hub 66, the interior of the inflatable seating element 64 together with the hub 66 in the location of the central seating section 114 define an annular inflation chamber 128 that is sealed to prevent escape of inflation fluid other than through the inflation port 126. The inflatable seating element 64 is made of a soft elastomeric material having a durometer in the range of approximately 10 A-20 such as silicone rubber. In one variation, the material has a high elongation in the range of approximately 400% to 1000%. The wall thickness of the inflatable seating element 64 is approximately one millimeter. The combination of the wall thickness and soft material with high elongation properties allows for controlled expansion of the inflatable seating element 64 in a balloon-like manner when the inflation chamber 128 is filled. When being filled with inflation fluid, the inflatable seating element 64 is configured to expand predominantly in a direction radial to the longitudinal axis relative to its expansion along the longitudinal axis. When maximally inflated, the outer diameter measured at the center of the inflatable portion 124 of the inflatable seating element 64 is approximately 6.0 cm.

The central inflatable portion 124 of the inflatable seating element 64, 164 includes a proximal surface 78 interconnected to a distal surface 80 at a curved circumferential outer edge 82. The proximal surface 78 and distal surface 80 are angled or curved such that the thickness of the central inflatable portion 124 increases toward the edge 82. A concave transitional surface 83 may be provided circumferentially between the proximal surface 78 and the proximal cylindrical portion 118. A similar concave transitional surface 83 may be provided between the distal surface 80 and the distal cylindrical portion 120.

The inflation chamber 128 is doughnut or ring shaped defining a central opening around the hub 66. The inflation port 126 provides fluidic connection between the inflation chamber 128 and the inflation tube 68. In one variation, the inflation tube 68 is removable and can be disconnected from the inflation port 126. A check valve may be provided in the inflation port 126 to prevent escape of inflation fluid from the inflation chamber 128 when the inflation tube 68 is removed. The inflation port 126 is radially distanced from the longitudinal axis such that the inflation port 126 and the shaft 28 are separated and offset from each other. The radial position of the inflation port 126 advantageously permits accessibility to easily attach and detach the inflation tube 68. The inflation tube 68 is supported by the channel 122 on the hub 66. The inflation port 126 rests in the channel 122 separated and distanced from the shaft 28 and advantageously allows for independent positioning, placement and securement relative to the shaft 28 during use. The channel 122 also serves as a tactile landmark for orientating the device 62 during insertion.

A fastener 60 is provided to secure the proximal end of the inflation tube 68 to the shaft 28. The inflation tube 68 may be as long as needed to connect to a source of inflation fluid via a connector 86. The inflatable seating element 64 is inflatable with inflation fluid via the inflation tube 68. The inflation fluid may be any suitable gaseous or liquid medium such as air or water provided in a reservoir for containing the inflation medium and a pump to move the inflation fluid from the reservoir/source to the inflation chamber.

The inflatable seating element 64 may be configured in a deflated state having a deflated configuration and an inflated state having an inflated configuration. In the deflated state, the pressure inside the inflation chamber 128 is neutral or atmospheric making the inflation chamber 128 softer, easily deflectable and compressible for comfortable and atraumatic insertion into and removal from the patient. Inflation fluid is delivered into the inflation chamber 128 and pressure inside the inflation chamber 128 increases.

In moving from the deflated configuration to the inflated configuration, the inflatable seating element 64 increases in rigidity and decreases in flexibility relative to the deflated configuration. The diameter of the inflatable portion 124 in the inflated configuration may increase relative to the deflated configuration. In one variation, the shape of the inflatable portion 124 when in the inflated configuration is substantially the same when in the deflated configuration. Air may be removed from the inflation chamber 128 via the inflation tube 68 to contract and reduce the size of the inflatable seating element 64 as needed. The inflatable portion 124 is more compliant in the deflated configuration. As such, the insertable device 62 is inserted into the vaginal canal when in the deflated configuration to facilitate insertion.

When the insertable device 62 is in the desired position, inflation fluid is delivered to inflation chamber 128. Pressure inside the inflatable seating element 64 increases anchoring the inflatable seating element 64 against the surrounding tissue creating a seal and/or blockage within the anatomy to facilitate creation and maintenance of a vacuum. A complete seal against the surrounding anatomy is not always achieved; however, a sufficient negative pressure may nonetheless be generated inside the uterus to suppress bleeding. Also, the inflatable seating element 64 need not be fully inflated to create a vacuum. A partially inflated inflatable seating element 64 may still advantageously facilitate generation and maintenance of a vacuum within the uterine cavity. Furthermore, when the vacuum is activated and a negative pressure is generated distal to the location of the seating element, a more complete seal is created as surrounding tissue collapses against the seating element due to vacuum formation. When in the inflated state, the edge 82 of the inflatable seating element 64 may press against and/or make a continuous radial contact with surrounding tissue of the vaginal canal or cervical wall so as to hold the inflatable seating element 64 in position to allow a vacuum to be created with the uterus. Vacuum is delivered to the uterus via the tip openings 34.

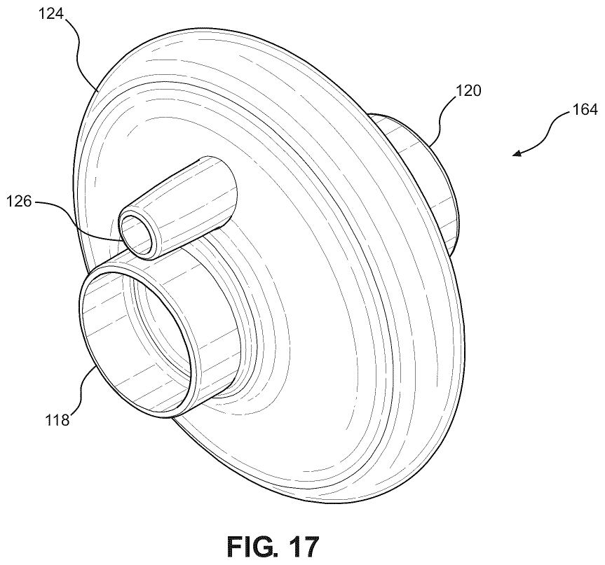

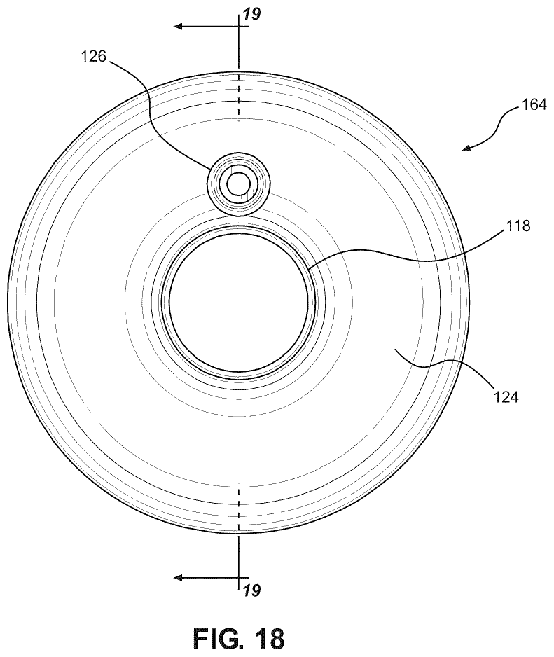

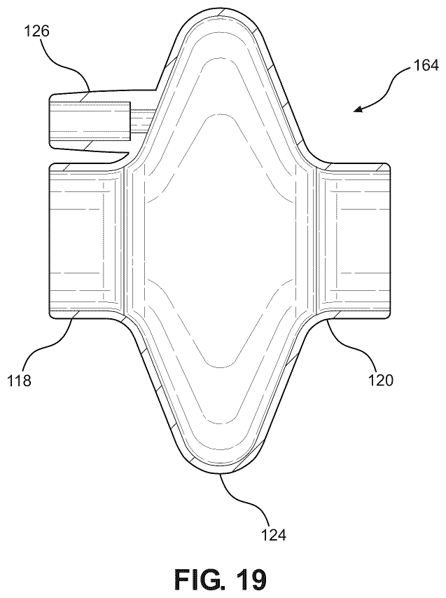

FIGS. 17-19 illustrate a variation of an inflatable seating element 164, wherein like reference numerals designate like components. The inflatable seating element 164 comprises a proximal cylindrical portion 118 and a distal cylindrical portion, interconnected by a central inflatable portion 124. An inflation port 126 is formed on the proximal side of the inflatable portion 124. Unlike the configuration shown in FIGS. 14-16, the inflation port 126 is radially spaced apart from, rather than conjoined with, the proximal cylindrical portion 118. The inflation port 126 extends in a proximal direction, substantially parallel to the proximal cylindrical portion 118, and is sized and configured to receive an inflation tube 68.

When connected, the inflation tube 68 is radially separated from the proximal cylindrical portion 118 at the inflation port 126. The spaced apart inflation tube 68 and inflation port 126 promotes more uniform and symmetrical inflation of the inflatable seating element 164. The inflatable seating element 164 mounts onto a hub in the same manner as previously described: the distal cylindrical portion 120 receives the distal seating section 110 of the hub and the proximal cylindrical portion 118 receives the proximal seating section 112 of the hub.

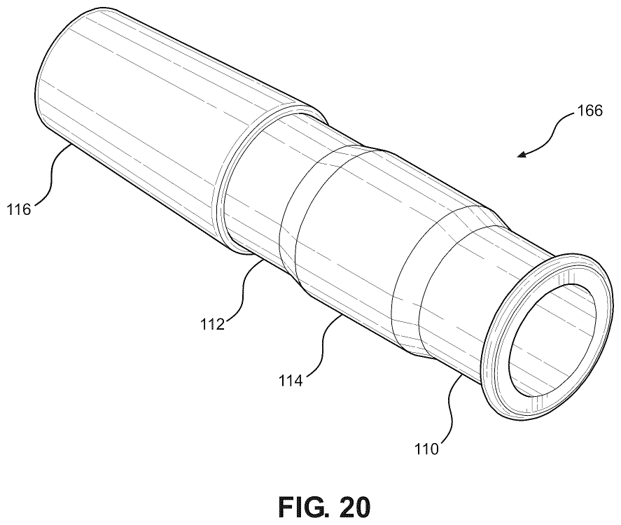

Because the inflation port 126 extends further radially and proximally relative to the inflatable seating element 64 of FIGS. 14-16, the inflation tube 68 is disposed at a greater distance from the tapered proximal portion 116 of the hub. Accordingly, a modified hub 166 without a channel 122 is employed, as shown in FIG. 20. The tapered proximal portion 116 of the hub 166 presents a continuous, smooth outer surface along its circumference. When assembled, the interior of the inflatable seating element 164, in conjunction with the central seating region of the hub 166, defines a sealed annular inflation chamber 128, configured to retain an inflation fluid except through the inflation port 126.

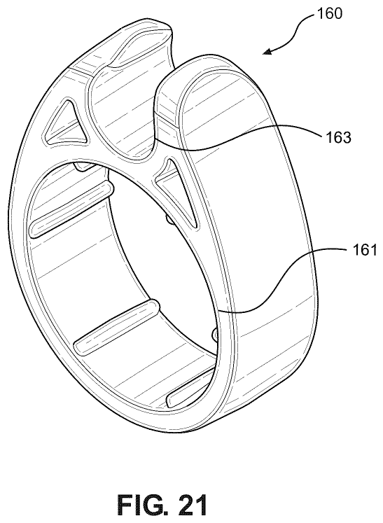

The proximal end of the inflation tube 68 includes a connector 86. Between the connector 86 and the inflation port 126, the inflation tube 68 is secured to the shaft 28 by a fastener 60. In certain embodiments, the fastener 60 comprises one or more strips of adhesive tape; in other embodiments, hook-and-loop tape may be used. In a further variation, the fastener 60 comprises a molded clip 160, shown in FIG. 21.

The clip 160 includes a circular opening 161 dimensioned to receive and retain the shaft 28, and a C-shaped opening 163 adjacent to the circular opening 161. The C-shaped opening 163 is configured for snap-fit engagement with the inflation tube 68, thereby retaining the inflation tube 68 securely while permitting slidable adjustment along the shaft 28. The clip 160 maintains the inflation tube 68 separated from the shaft 28 and in an extended orientation along its length. This configuration is positioned proximally near the connector 86 to optimize tube routing. The clip 160 can be employed with any of the inflatable seating element embodiments described herein.

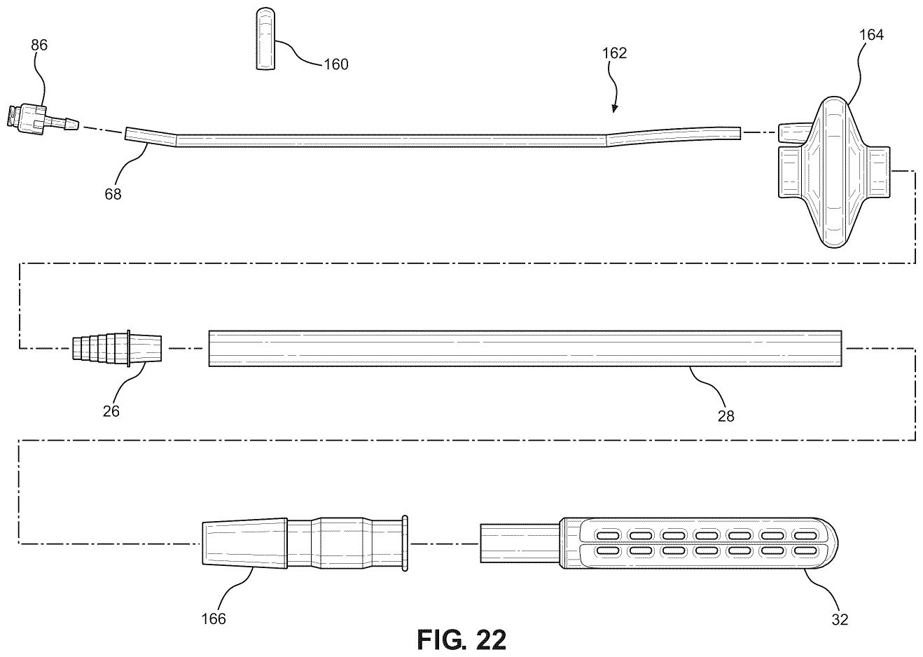

FIG. 22 illustrates another embodiment of an insertable device 162, generally similar to the insertable device 62 of FIGS. 10-16, wherein like reference numerals denote like elements. The insertable device 162 comprises a shaft 28 with a connector 26 at its proximal end. The distal end of the shaft 28 is coupled to the proximal end of the hub 166, while the distal end of the hub 166 is coupled to a tip 32. The inflatable seating element 164 is mounted to the hub 166 and in fluid communication with the inflation tube 68, which terminates in a proximal connector 86. A clip 160 secures the proximal portion of the inflation tube 68 along the shaft 28.

The connector 26 at the proximal end of the shaft 28 is connected to a source of negative pressure such as a pump via connection tubing. The insertable device 162 is inserted transvaginally such that the tip 32 is positioned within the uterus, inflation fluid is delivered into the inflatable seating element 164 and expanded to contact vaginal anatomy and vacuum is delivered from the source of negative pressure through the connection tubing, shaft 28, hub 66, and the tip 32 to arrest uterine hemorrhaging.

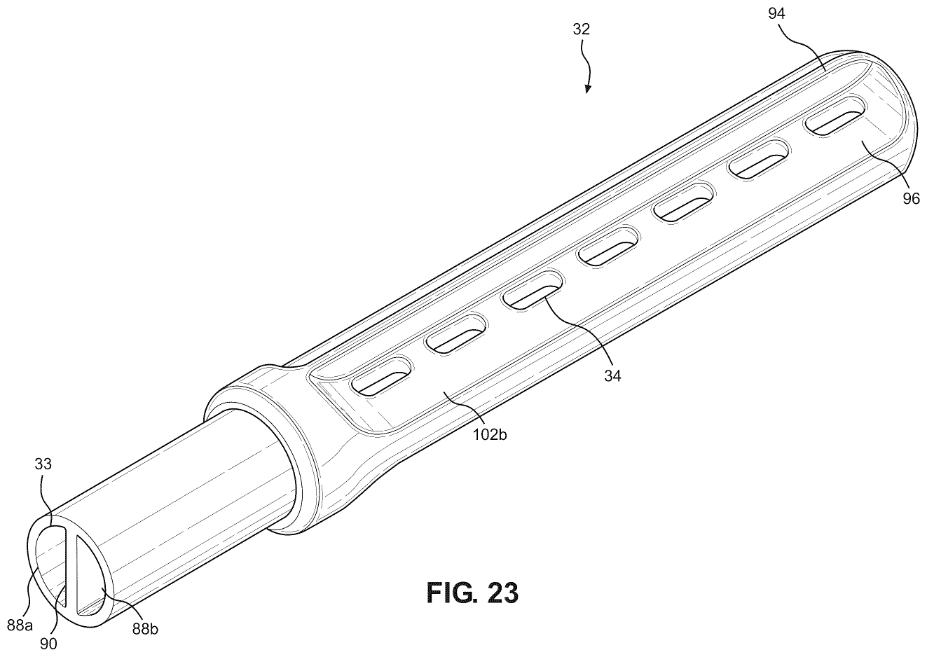

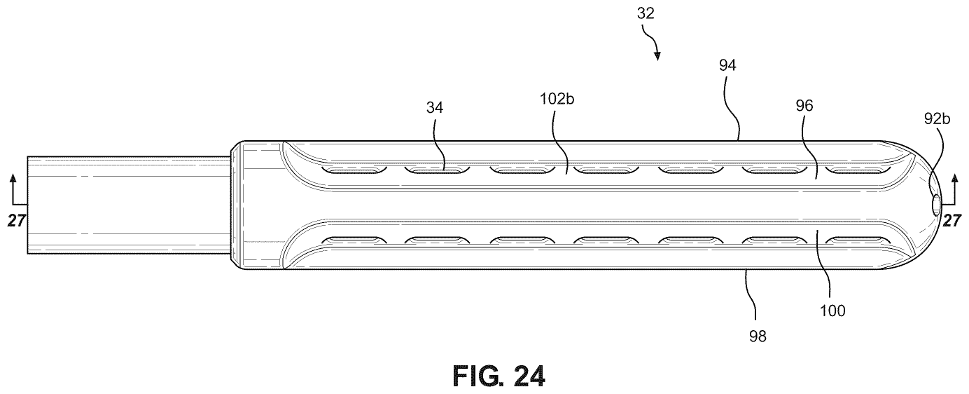

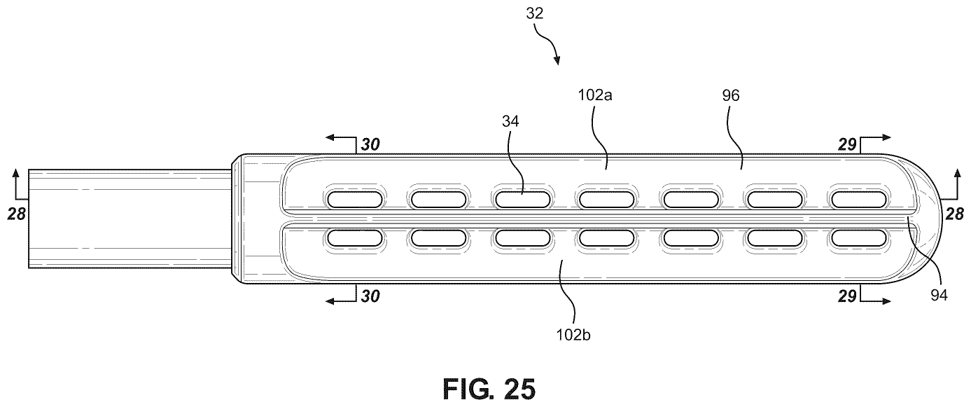

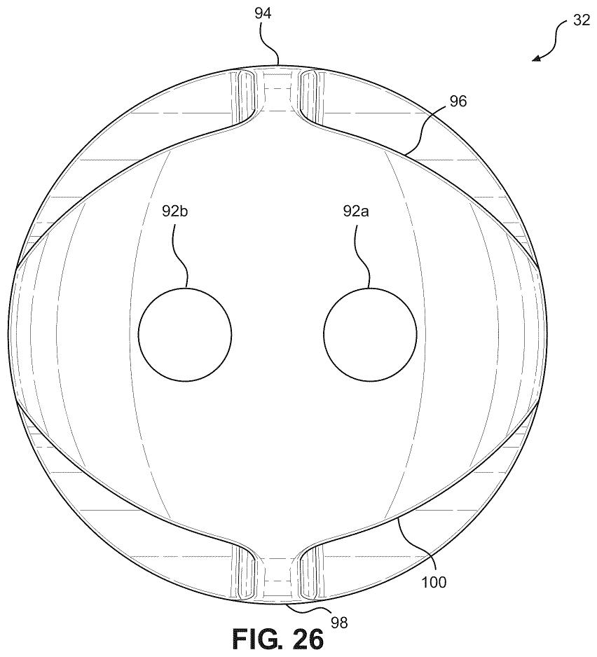

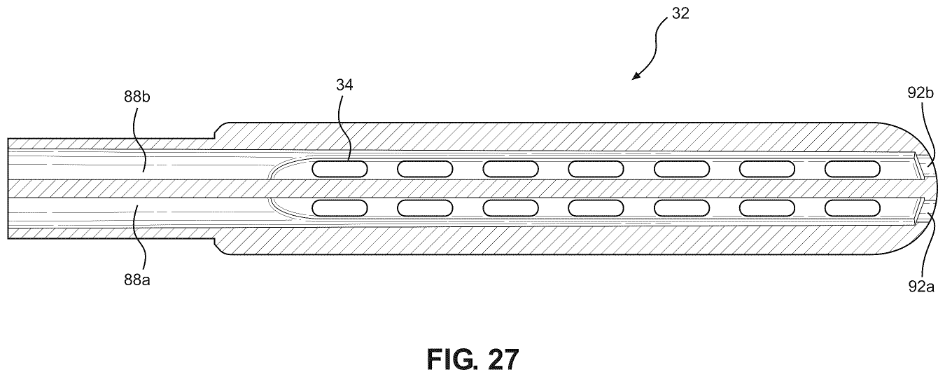

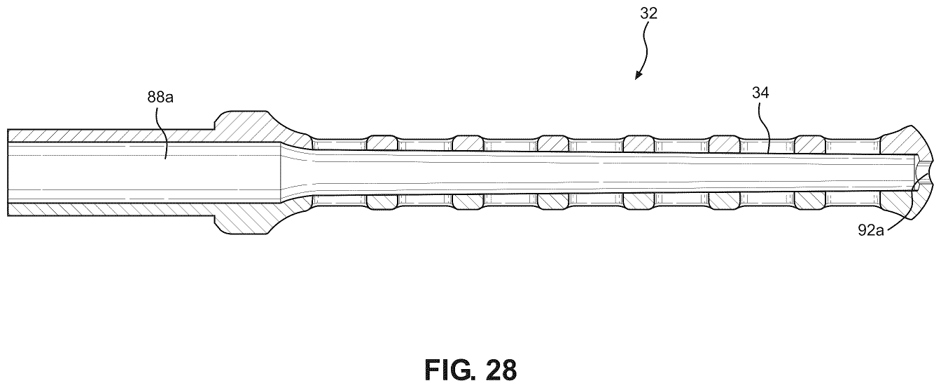

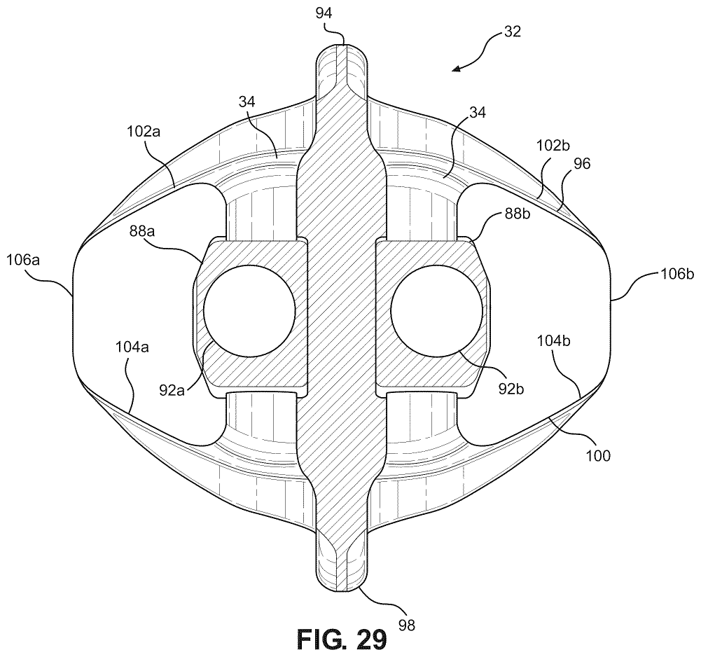

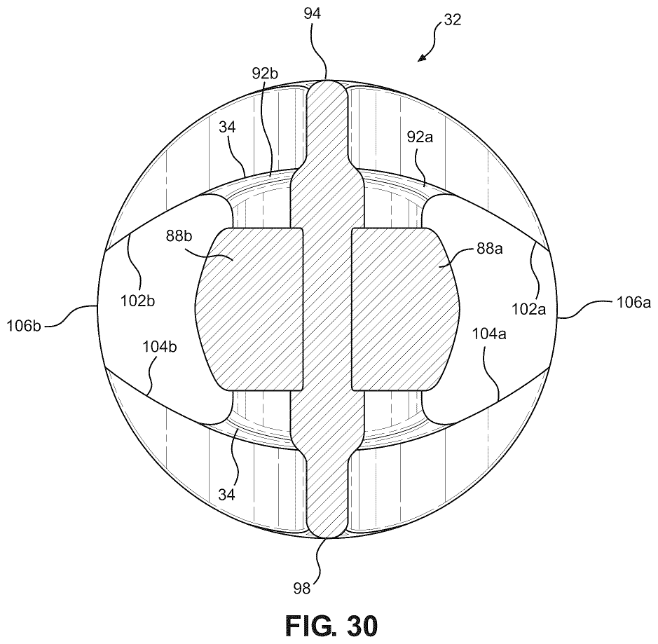

FIGS. 23-30 illustrate a tip 32 according to one or more embodiments that can be employed with either of the insertable devices 12, 62, 162 described above. The tip 32 includes a tip lumen 33 extending along the longitudinal axis. The tip lumen 33 includes a septum wall 90 extending in a radial plane parallel to the longitudinal axis along the length of the tip 32. In one variation, a separate septum wall extends throughout the length of the shaft lumen 30. In another variation, the septum wall 90 extends only along a length of the tip 32. The septum wall 90 divides the tip lumen 33 into two separate tip channels 88a, 88b (e.g., tip lumens) extending parallel to the longitudinal axis. The tip channels 88a, 88b open to respective circular openings 92a, 92b formed in a distal surface at the distal end of the tip 32. The tip channels 88a, 88b have a D-shaped cross-section. Tip channels 88a, 88b advantageously provide a redundancy in evacuation channels for fluid and debris to escape. This redundancy for material evacuation advantageously permits continued functionality of negative pressure generation and fluid/debris removal at the uterus under conditions in which there is reduced performance in either of the channels 88a, 88b such as may arise from debris blockages or fluctuations in pressure generation.

The tip 32 includes a top rib 94 extending radially outwardly from a top surface 96 and a bottom rib 98 extending outwardly from a bottom surface 100. The top and bottom ribs 94, 98 are aligned with each other and, in one variation, also aligned with the septum wall 90 in a vertical radial plane that includes the longitudinal axis. The top and bottom ribs 94, 98 extend from a proximal end of the tip 32 to a distal end of the tip 32 in the location of the tip openings 34. The top and bottom ribs 94, 98 together with the septum wall 90 provide structural rigidity and maintain patency of the tip channels 88a, 88b when the tip 32 is bent. As a result, the tip 32 may flex more readily in a side-to-side direction relative to an up-and-down direction.

The top rib 94 divides the top surface 96 into two top surfaces 102a, 102b and the bottom rib 98 divides the bottom surface 100 into two bottom surfaces 104a, 104b. The tip openings 34 may be formed in the top surfaces 102a, 102b and bottom surfaces 104a, 104b in a location as close as possible to the top and bottom ribs 94, 98 relative to where the top surfaces 102a, 102b and bottom surfaces 104a, 104b meet the outer perimeter of tip 32. Also, the tip openings 34 do not extend radially all the way to the side perimeter. For example, the tip openings 32 may be formed in the top and bottom surface 96, 100 to be positioned closer to the top and bottom ribs 94, 98 than to the side perimeter. As a result, the top and bottom ribs 94, 98 advantageously baffle the tip openings 34 from surrounding tissue to keep the tip openings 34 clear from becoming obstructed when vacuum is applied.

The two top surfaces 102a, 102b are substantially perpendicular to the top rib 94 and curve down away from the top rib 94 toward the side outer perimeter to advantageously extend the surrounding area to which vacuum is applied at the top end of the tip 32. Similarly, the two bottom surfaces 104a, 104b are substantially perpendicular to the bottom rib 98 and curve up away from the bottom rib 98 toward the side outer perimeter to advantageously extend the surrounding area to which vacuum is applied at the bottom of the tip 32. Extending between the top surface 102a and the bottom surface 104a is a first side surface 106a and between the top surface 102b and the bottom surface 104b is a second side surface 106b. The top rib 94 and the bottom rib 98 extend to an outer perimeter wherein together with the first side surface 106a and second side surface 106b, the tip 32 defines a generally circular profile in a plane perpendicular to the longitudinal axis wherein the circular profile includes the distal end and proximal ends of the tip 32.

The tip 32 transitions to a reduced diameter at the proximal end that is sized and configured for insertion into the distal end of the hub 66 or seating element. The transition defines a chamfered edge to prevent a pinch point in the device. The tip 32 is made of a soft elastomeric material having a durometer in the range of approximately 40-60 A such as silicone rubber, thermoplastic elastomer or thermoplastic urethane. The soft material prevents tissue trauma while providing sufficient flexibility and stiffness for usability.

The tip openings 34 form a row of openings 34 (e.g., a longitudinally extending series of tip openings 34) in each of the two top surfaces 102a, 102b, the two bottom surfaces 104a, 104b, thereby, placing the openings in four quadrants around the tip 32 to advantageously provide a 360-degree area of vacuum application. Each row is shown to have seven tip openings 34 for a total of 28 holes around the tip 32, with two additional openings 92a, 92b at the distal end; however, the invention is not so limited and any number of tip openings 34 and rows are within the spirit and scope of the invention. The high number of holes and their separation into four quadrants advantageously prevent full occlusion and loss of suction and flow rate. The tip openings 34 in the top surface 102a and bottom surface 104a are in fluidic communication with the first D-shaped channel 88a and the tip openings 34 in the top surface 102b and bottom surface 104b are in fluidic communication with the second D-shaped channel 88b. The openings 34 are elongated in shape and the distal end of the tip 32 is rounded and atraumatic.

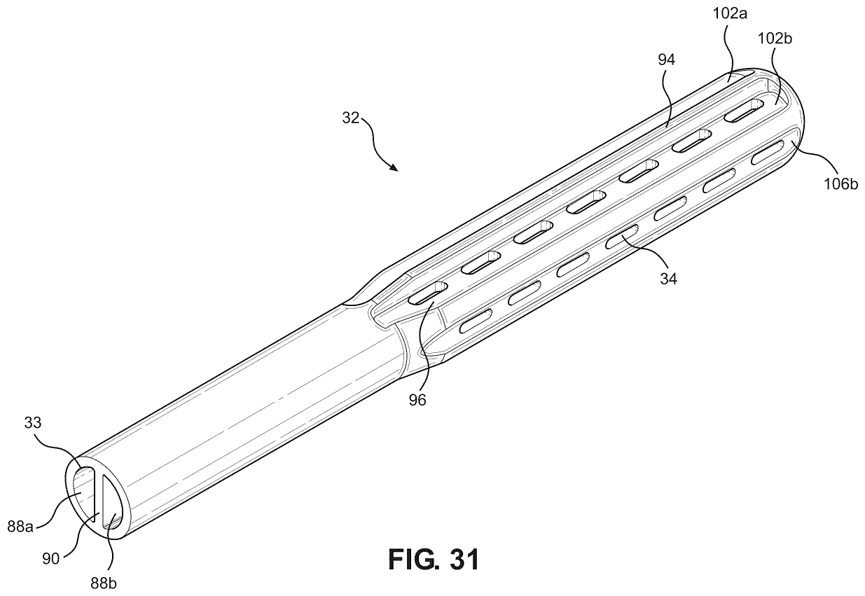

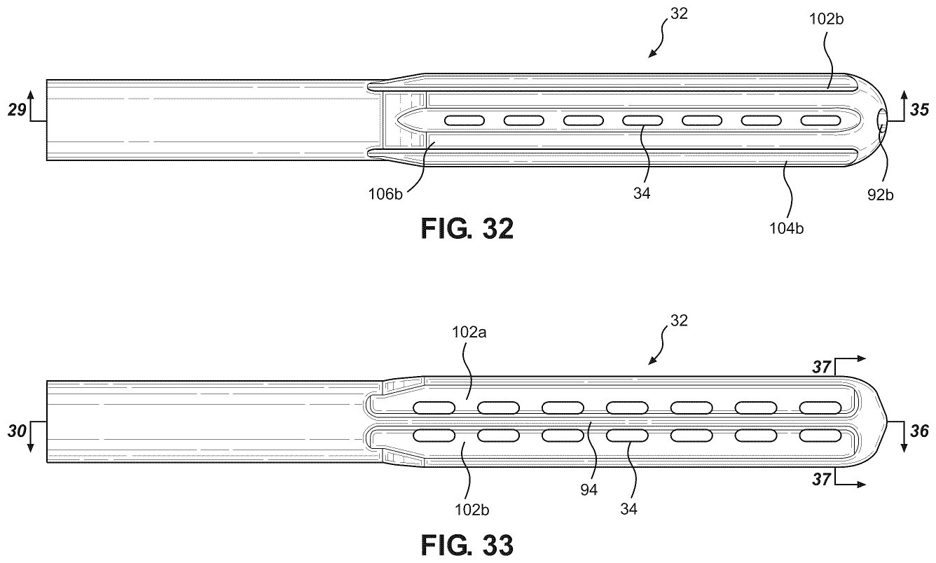

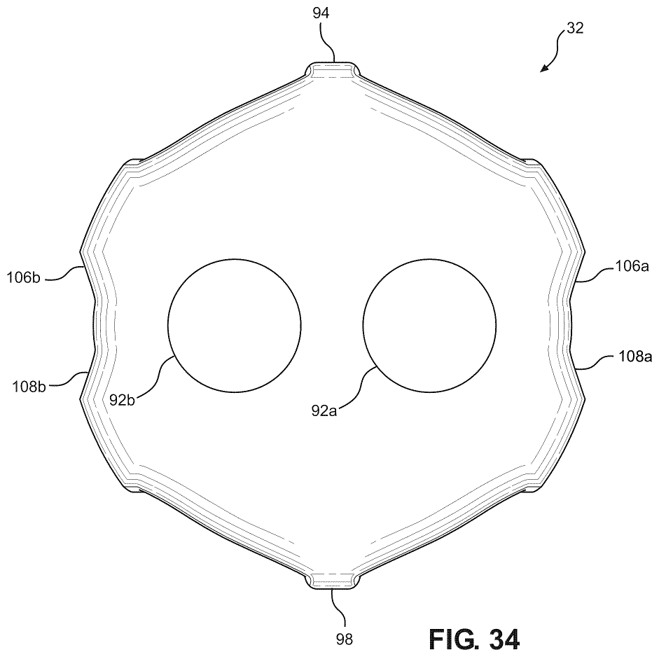

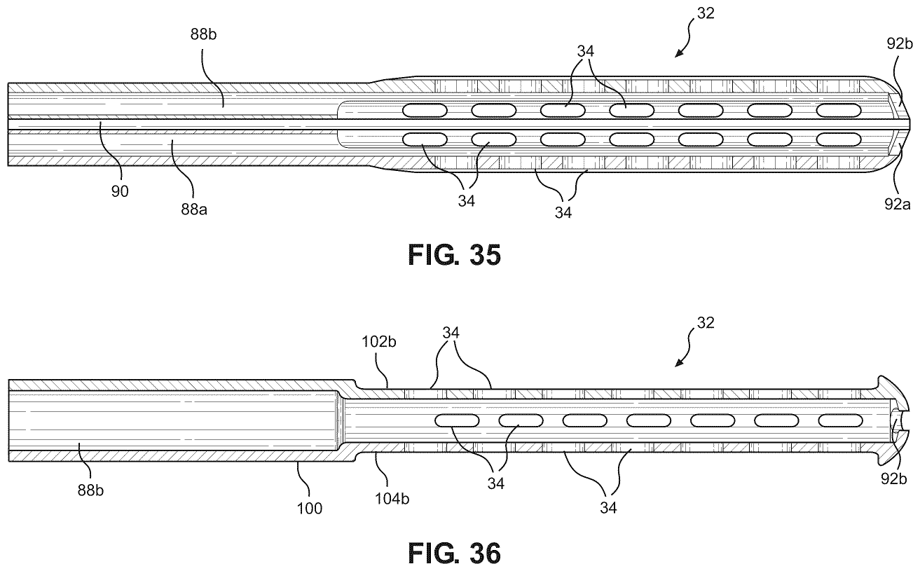

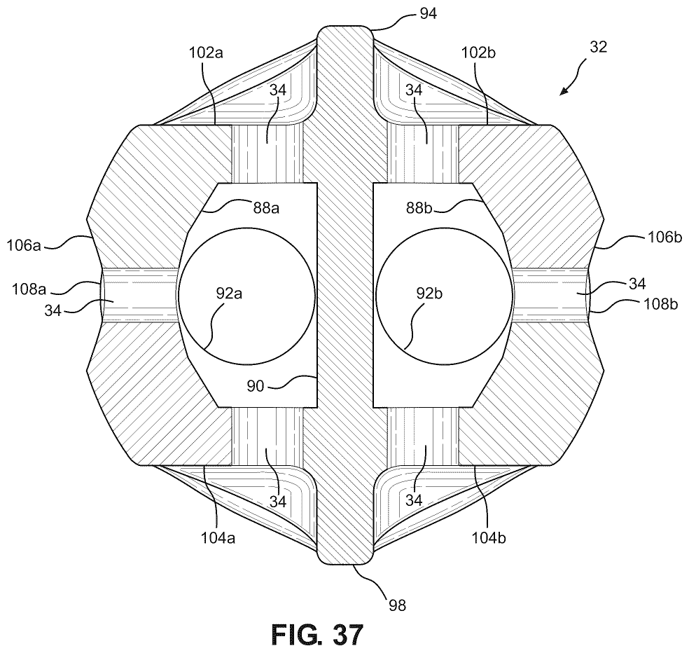

FIGS. 31-37 illustrate another variation of a tip 32 for the insertable device 12, 62 that is similar to the tip 32 shown in FIGS. 23-30 wherein like reference numbers are used to describe like parts. The tip 32 includes a tip lumen 33 extending along the longitudinal axis. The tip lumen 33 includes a septum wall 90 extending in a radial plane parallel to the longitudinal axis along the length of the tip 32. In one variation, a septum wall also extends throughout the length of the shaft lumen 30. In another variation, the septum wall 90 extends only along a length of the tip 32. The septum wall 90 divides the tip lumen 33 into two separate tip channels 88a, 88b. The tip channels 88a, 88b have a D-shaped cross-section. The tip channels 88a, 88b interconnect with circular openings 92a, 92b, respectively, at the distal end of the tip 32.

The tip 32 includes a top rib 94 extending radially outwardly from a top surface 96 and a bottom rib 98 extending outwardly from a bottom surface 100. The top and bottom ribs 94, 98 are aligned with each other and, in one variation, also aligned with the septum wall 90 in a vertical radial plane that includes the longitudinal axis. The top and bottom ribs 94, 98 extend from a proximal end of the tip 32 to a distal end of the tip 32. The top rib 94 divides the top surface 96 into two top surfaces 102a, 102b and the bottom rib 98 divides the bottom surface 100 into two bottom surfaces 104a, 104b. The tip openings 34 are formed in the top surfaces 102a, 102b and bottom surfaces 104a, 104b in a location as close as possible to the top and bottom ribs 94, 98 relative to where the top surfaces 102a, 102b and bottom surfaces 104a, 104b meet the outer perimeter of tip 32. Thereby, the top and bottom ribs 94, 98 advantageously baffle the tip openings 34 from surrounding tissue to keep the tip openings 34 clear from becoming obstructed when vacuum is applied.

The two top surfaces 102a, 102b are substantially perpendicular to the top rib 94 and curve down away from the top rib 94 to advantageously extend the surrounding area to which vacuum is applied at the top end of the tip 32. Similarly, the two bottom surfaces 104a, 104b are substantially perpendicular to the bottom rib 98 and curve up away from the bottom rib 98 to advantageously extend the surrounding area to which vacuum is applied at the bottom of the tip 32.

Extending between the top surface 102a and the bottom surface 104a is a first side surface 106a and between the top surface 102b and the bottom surface 104b is a second side surface 106b. The first side surface 106a includes a first side concavity 108a and the second side surface 106b includes a second side concavity 108b. The first and second side concavities 108a, 108b are centered on a horizontal radial plane that is perpendicular to the vertical radial plane. The tip 32 defines a generally circular profile in a plane perpendicular to the longitudinal axis wherein the circular profile includes the distal end and proximal ends of the tip 32. The tip 32 transitions to a reduced diameter at the proximal end that is sized and configured for insertion into the distal end of the hub 66 or seating element. The transition defines a chamfered edge to prevent a pinch point in the device. The tip 32 is made of a soft elastomeric material having a durometer in the range of approximately 40-60 A such as silicone rubber, thermoplastic elastomer or thermoplastic urethane. The soft material prevents tissue trauma while providing sufficient flexibility and stiffness for usability.

The plurality of tip openings 34 are arranged in rows in each of the two top surfaces 102a, 102b, the two bottom surfaces 104a, 104b, the first side concavity 108a, and the second side concavity 108b. The tip openings 34 in the top surface 102a, bottom surface 104a and first side concavity 108a are in fluidic communication with the first D-shaped channel 88a and the tip openings 34 in the top surface 102b, bottom surface 104b, and the second side concavity 108b are in fluidic communication with the second D-shaped channel 88b. The openings 34 are elongated in shape and the distal end of the tip 32 is rounded and atraumatic. There are approximately seven openings 34 in each of the top and bottom surfaces 102a, 102b, 104a, 104b and in each of the side surfaces 106a, 106b. Six rows of seven tip openings 34 are arranged in six quadrants around the tip 32 providing 360 degrees of vacuum application around the tip 32. The top rib 94 and bottom rib 98 along with the openings 34 being located directly adjacent at the base of the ribs 94, 98 advantageously prevent the tip openings 34 formed in the top surface 102 and bottom surface 104, respectively, from becoming obstructed with tissue and/or debris. Also, the concavity of the side surfaces 106a, 106b shelter the tip openings 34 long the sides from obstruction with tissue and debris during application of negative pressure.

To assemble the insertable device 12, 62, 162, a tip 32 is connected to the distal end of a seating element 22, 64, 164 either directly or via a hub 66, 166 which in turn is connected to the distal end of the shaft 28. The proximal end of the shaft 28 is connected to a vacuum source via the connector 26. If an insertable device 62, 162 having an inflatable seating element 64, 164 is employed the inflation tube 68 is connected to a source of inflation fluid via the connector 86.

The assembly of the insertable device 12, 62, 162 advantageously employs adhesive bonding to secure selected components, thereby enhancing the structural integrity and reliability of the device. Adhesive may be utilized to bond the seating element 22 to the shaft 28 and tip 32; the inflatable seating element 64, 164 to the hub 66, 166; the hub 66, 166 to the shaft 28; the inflation tube 68 to the inflatable seating element 64, 164; the hub 66 to the connector 26; and the inflation tube 68 to the connector 86. In particular, adhesive may be applied to attach the inflatable seating element 64, 164 to both the distal and proximal seating sections 110, 112 of the hub 66, 166. The distal cylindrical portion 120 of the inflatable seating element defines a distal opening sized and configured to receive and encompass the distal seating section 110 of the hub 66, 166, and is bonded thereto with adhesive for a secure connection. Similarly, the proximal cylindrical portion 118 defines a proximal opening sized and configured to receive and encompass the proximal seating section 112 of the hub 66, 166, and is affixed to said section using adhesive. This approach ensures robust attachment and fluid-tight interfaces throughout the assembled device.

The term “adhesive” as used herein encompasses any material suitable for forming a functional bond between at least two surfaces or device components. Representative adhesives include room temperature vulcanization (RTV) compositions, ultraviolet curing (UV curing) adhesives, epoxy resins, cyanoacrylate formulations, and other biocompatible adhesives suitable for medical devices. Adhesives may be optionally used in conjunction with primers to facilitate bonding to substrates, such as silicone, polycarbonate, or other medical-grade plastics.

In one embodiment, assembly of the device is achieved with a room temperature vulcanization (RTV) process. RTV silicones, available as single-component or dual-component systems, cure and bond device components at ambient conditions over a period of several hours, forming durable, elastic joints with excellent resistance to aging, ultraviolet exposure, and sterilizing environments.

In a preferred embodiment, ultraviolet curing (UV curing) adhesive is employed to bond components. UV-curing adhesives offer several key advantages in medical device manufacturing, including accelerated cure time (reduction from hours for RTV to minutes for UV curing), precision application, biocompatibility, strong and sterilizable bonds, and minimal environmental impact due to lower solvent content. Curing is initiated by exposure to ultraviolet light at ambient or slightly elevated temperatures, with completion in seconds or minutes. Use of UV-curable adhesives enables increased production throughput and reduction in manufacturing costs.

A specific embodiment utilizes ultraviolet curing (UV curing) adhesive for assembly of the inflatable seating element, hub, inflation tube, and related components. This method results in shorter production times, reduced manufacturing expense, consistent and precise assembly, and durable, sterilizable joints suitable for medical applications. Primers may be optionally applied to improve bond strength and reliability, particularly for challenging materials.

Additional adhesive options for device construction may include epoxy resins, which provide high-strength and chemical-resistant bonds, and cyanoacrylate adhesives, which offer rapid bonding for various medical materials, especially when used with compatible primers. Adhesive selection is determined by the compatibility with device materials, required bond strength, sterilization compatibility, and compliance with biocompatibility standards relevant to medical device use.

In use, the device 12 of FIGS. 1-9 is held by the user at the proximal end of the shaft 28. The tip 32 is inserted into the vaginal canal such that the tip 32 is positioned inside the uterus. During insertion of the device 12, the atraumatic funnel-like seating element 22 will deflect against tissue due to the resilience of the seating element material and rib geometry, the seating element 22 will spring back towards its original shape, remain deflected, and/or invert to contact surrounding tissue for sealing engagement therewith. Any one or more of the distal surface 48, proximal surface 50, edge 52 and bead 54 of the seating element 22 may contact the surrounding tissue to facilitate establishing a vacuum chamber distal to the seating element 22 within the uterus. In addition to the seating element 22, the shaft 28 and tip 32 are flexible and atraumatic. The ridge 44 provides a location for finger placement and tactile feedback to help the user gently guide the device into position. The device 12 may be retracted and repositioned if needed to create a sealing engagement of the seating element 22 with the surrounding tissue. When in position, the tip 32 will reside in the uterus and the seating element 22 will contact tissue proximal to the tip openings 34.

If any of the devices 62, 162 of FIGS. 10-22 is employed, the inflatable seating element 64, 164 is preferably placed into a deflated configuration for insertion of the device 62, 162. In the deflated state, the inflatable seating element 64, 164 is not under pressure and is capable of deflecting, bending and conforming against tissue while being inserted. When the device 62, 162 is in position, inflation fluid, such as air or liquid, is delivered from a source into the inflatable seating element 64, 164 via the inflation tube 68 that is connected to a source of inflation fluid. Pressure inside the inflatable seating element 64, 164 will increase to achieve an inflated configuration or partially inflated configuration in which the inflatable seating element 64, 164 is more rigid relative to the deflated state and, as a result, will press against and conformingly seal against the surrounding tissue. The desired fluidic pressure inside the inflatable seating element 64, 164 is maintained. The inflation tube 68 is flexible and separate from the shaft 28 and, therefore, can be manipulated independently from the shaft 28 for insertion and delivery of inflation fluid. Also, the inflation port 126 and connector 86 for the inflation tube 68 are located radially away from the shaft 28. Therefore, the inflation tube 68 may be moved advantageously away from sensitive patient anatomy for improved patient comfort.

When either of the devices 12, 62, 162 are in position, the vacuum is activated and delivered through the shaft 28 and to the uterus via the tip openings 34. The vacuum will draw air and fluid from the uterus through the device 12, 62, 162 out of the patient and into a collection container. The negative pressure is maintained via the seating element 22, 64, 164 and can be measured by a gauge outside of the patient. Collection of fluid and air may be observed through a transparent shaft, collection container, and/or connection tubing. Air and fluid from the uterus will be drawn into the device 12, 62, 162 through the tip openings 34. Tissue, debris and fluid may enter the tip lumen 33 and proceed into the shaft lumen 30 on its way to the collection container. The tip lumen 33 and/or the shaft lumen 30 is advantageously bifurcated into two channels 88a, 88b via a septum wall 90. In the event one of the channels 88a, 88b is obstructed by tissue or other debris and fluid, vacuum can still be applied, and fluid and air extracted via the other channel. Furthermore, as vacuum is applied and negative pressure is established inside the uterus, the uterine walls may collapse around and onto the tip 32. The top rib 94 and the bottom rib 98 advantageously serve to prevent the collapsing uterine walls from obstructing the tip openings 34 that are located adjacent to the ribs 94, 98. As a result of tip openings 34 being located in the longitudinally extending recesses of the side concavities 108a, 108b, obstruction of these side tip openings 34 by collapsing uterine walls is also avoided. Vacuum is applied and extraction of fluids is further advantageously facilitated not only by recessed side openings 34 and rib-protected top and bottom openings but also by two distal tip openings 92a, 92b. All of the tip openings 34 provide multi-directional vacuum application and fluid extraction ability while maintaining an even pressure as much as possible 360 degrees around the circumference of the tip 32. Furthermore, the elongated shape of each tip opening 34, the major axis of which is aligned with the longitudinal axis, minimizes the ease with which an opening 34 may be obstructed.

When the procedure is completed, the device 12, 62, 162 is removed from the patient. If a device 12 with the funnel-like seating element 22 is employed, the flexible seating element 22 may deflect/invert as the device 12 is moved proximally. If a device 62, 162 with the inflatable seating element 64, 164 is employed, the inflatable seating element 64, 164 is deflated by removing inflation fluid from the inflation chamber. When the inflatable seating element 64, 164 is in a deflated state, the device 62, 162 is moved proximally for removal. When in the deflated configuration, the relatively more flexible seating element 64, 164 will easily deflect against tissue as it is removed.

Those skilled in the art will appreciate from the foregoing description that the broad techniques of the examples of the present disclosure can be implemented in a variety of forms. Therefore, while the examples of this disclosure have been described in connection with particular examples thereof, the true scope of the examples of the disclosure should not be so limited since other modifications will become apparent to the skilled practitioner upon a study of the drawings, specification, and following claims.

We claim:

1. A device, comprising:
a shaft having a shaft lumen extending along a longitudinal axis;
an elongated tip connected to a distal end of the shaft such that a tip lumen is in fluidic communication with the shaft lumen; the elongated tip extending from a proximal end to a distal end and having a plurality of tip openings connected to the tip lumen; and
a seating element connected to the shaft at a location proximal to the plurality of tip openings of the elongated tip and extending circumferentially around the shaft and radially outward from the shaft.

2. The device of claim 1,
wherein the tip lumen is divided into two separate tip lumens extending from the proximal end of the elongated tip to the distal end of the elongated tip; and
wherein a portion of the plurality of tip openings are fluidly interconnected with one of the two separate tip lumens and a remainder portion of the plurality of tip openings are fluidly interconnected with the other one of the two separate tip lumens.

3. The device of claim 2, wherein each of the two separate tip lumens aligns and connects with a respective distal opening at the distal end of the elongated tip.

4. The device of claim 1, wherein the diameter of the seating element is larger than a diameter of the shaft and larger than a diameter of the elongated tip.

5. The device of claim 1, wherein the plurality of tip openings is arranged in rows, each row extending longitudinally along the elongated tip and arranged around a perimeter of the elongated tip.

6. The device of claim 1,
wherein the elongated tip includes a top rib extending radially outwardly from a top surface and a bottom rib extending radially outwardly from a bottom surface;
wherein the top rib divides the top surface into a first top surface and a second top surface, each of the first and second top surfaces having a row of the plurality of tip openings; and
wherein the bottom rib divides the bottom surface into a first bottom surface and a second bottom surface, each of the first and second bottom surfaces having a row of the plurality of tip openings.

7. The device of claim 6, wherein each row of the plurality of tip openings is located closer to the top rib or bottom rib than to an outer perimeter of the device.

8. The device of claim 6, wherein the top rib is radially aligned with the bottom rib.

9. The device of claim 6 further including a first side surface and a second side surface interconnecting the top surface and the bottom surface, each of the first side surface and second side surface including a row of the plurality of tip openings.

10. The device of claim 9, wherein the first side surface and the second side surface are concave such that the row of the plurality of tip openings in each of the side surfaces is recessed.

11. The device of claim 8 further including a septum wall dividing the tip lumen into a first tip lumen and a second tip lumen, wherein the tip openings in the first top surface, first side surface, and first bottom surface are in fluidic communication with the first tip lumen and the tip openings in the second top surface, second side surface and second bottom surface are in fluidic communication with the second tip lumen.

12. The device of claim 1, wherein the seating element is an inflatable seating element that includes an inflatable portion having an inflation port radially offset from the shaft, the inflation port connectable to an inflation tube extending proximally from the inflation port.

13. The device of claim 1 further including a hub having a hub lumen defining a proximal portion configured to receive the shaft and a distal portion configured to receive the elongated tip, wherein the seating element is an inflatable seating element that includes an inflatable portion mounted to an outer surface of the hub.

14. The device of claim 13, wherein a proximal portion of the hub is tapered to form a smooth transition with the shaft.

15. The device of claim 13, wherein the distal portion of the hub includes a circumferential ridge having an increased diameter configured to provide tactile feedback to a user for guiding the device.

16. The device of claim 1, wherein the seating element includes a flexible flared portion having a convex distal surface interconnected to a concave proximal surface at a circumferential edge, the concave proximal surface having a plurality of radially extending ribs having an increased thickness.

17. The device of claim 16, wherein a thickness of the flared portion at a cross-section through a rib taken perpendicular to the longitudinal axis of the shaft decreases radially outwardly from the longitudinal axis.

18. The device of claim 16 further including a bead around a perimeter of the flared portion, wherein the thickness increases at the bead.

19. The device of claim 16, wherein the flared portion has an unbiased configuration and an inverted configuration.

20. The device of claim 16, wherein the seating element includes a stem having a stem lumen defining a proximal portion configured to receive the shaft and a distal portion configured to receive the elongated tip, and a transition portion between the proximal portion and distal portion, wherein the flared portion extends radially outwardly from an outer surface of the stem.

21. The device of claim 12 further comprising a fastener securing the inflation tube to the shaft.

22. The device of claim 21, wherein the fastener comprises one or more strips of adhesive tape.

23. The device of claim 21, wherein the fastener comprises one or more strips of hook-and-loop tape.

24. The device of claim 21, wherein the fastener comprises a molded clip.

25. The device of claim 24, wherein the molded clip comprises a circular opening configured to receive the shaft and a C-shaped opening for snap-fit engagement of the inflation tube.

26. The device of claim 24, wherein the molded clip is slidable along the shaft to maintain the inflation tube in an extended configuration and separated from the shaft.

27. The device of claim 13, wherein the inflatable seating element is mounted to the hub using ultraviolet light curing adhesive.

28. An insertable device comprising:
an elongated shaft defining a shaft lumen;
a suction tip at a distal portion of the insertable device, the suction tip defining a tip lumen in fluid communication with the shaft lumen and having a plurality of tip openings in fluid communication with the tip lumen; and
a seating element disposed proximal to the plurality of tip openings and configured to engage patient anatomy to facilitate creation of a reduced-pressure zone in a uterine cavity when the suction tip is positioned in the uterine cavity.

29. A uterine hemorrhage controlling device, comprising:
an elongated shaft defining a shaft lumen and having a proximal connector configured to couple to a source of negative pressure;
a suction tip defining a tip lumen in fluid communication with the shaft lumen and having a tip opening in fluid communication with the tip lumen;
a seating element extending circumferentially radially outwardly around the shaft and radially outward from the shaft to facilitate creation of a reduced-pressure zone in a uterine cavity when the suction tip is positioned in the uterine cavity.
